(19) United States
(12) Patent Application Publication
(10) Pub. No.: US 2005/0056551 A1
(43) Pub. Date: Mar. 17, 2005

(54) Electrochemical detection of analytes

(75) Inventors: Stephen Fredrick White, Kempston (GB); John Anthony Bolbot, Milton (GB)

Correspondence Address: OBLON, SPIVAK, MCCLELLAND, MAIER & NEUSTADT, P.C., 1940 DUKE STREET, ALEXANDRIA, VA, 22314, US

(73) Assignee: Cranfield University

(21) Appl. No.: 10/482,334

(22) PCT Filed: Jun. 28, 2002
(86) PCT No.: PCT/GB02/03004

(30) Foreign Application Priority Data
Jun. 28, 2001 (GB) 0115793.2

(51) Int. Cl. G01N 1/00; G01N 33/50

(52) U.S. Cl. 205/777.5; 204/403.01; 204/403.1

(57) ABSTRACT

An analyte is determined electrochemically indirectly. In a first step an oxidase and an oxidisable substrate, one of which is the analyte or derived therefrom, interact and generate hydrogen peroxide. In a second step a peroxidase (especially horse-radish peroxidase) reduces the hydrogen peroxide and concomitantly oxidises a mediator, preferably 2,2′-azino-bis(3-ethyl=benzthiazoline-6-sulfonic acid, ABTS) to an oxidised form (ABTSOX). The oxidised mediator is then reduced at an electrode and the consequent current is measured. A preferred sensor format uses a carbon electrode screen-printed onto a substrate and overlaid with one or more layers containing the enzymes and other components.

### TECHNICAL FIELD

The present invention relates to the electrochemical detection of analytes, particularly analytes of biological/medical significance such as cholesterol. Different aspects relate to a method, a sensor device suitable for use in the method, and the manufacture of such devices.

The main cause of death in developed countries is cardiovascular disease and the contribution of elevated blood cholesterol levels to this is well established. There is consequently a need to measure these levels (physiological range 150-250+ mg/dL) in order to diagnose the condition & prescribe appropriate dietary or pharmaceutical treatment. In one group of embodiments, this invention consists of the adaptation of a cholesterol colour test to an electrochemical test.

### DISCLOSURE OF INVENTION

In a first aspect the invention provides a method of detecting an analyte wherein the analyte is either (a) a substrate which is oxidisable by means of an oxidase with the generation of hydrogen peroxide, the quantity of hydrogen peroxide being dependent on the quantity of analyte, or said analyte is convertible into a said oxidisable substrate, or (b) said oxidase; said method of comprising.
(a) providing component A which comprises (a) said oxidase if the analyte is said oxidisable substrate, or (b) said oxidisable substrate if the analyte is said oxidase; providing component B which comprises a peroxidase capable of oxidising a mediator comprising 2,2′-azino-bis-(3-ethylbenzthiazoline-6-sulphonic acid) (“ABTS”) with concomitant reduction of hydrogen peroxide; and providing an electrochemical cell comprising a redox electrode connected to a counter-electrode;
(b) allowing a sample suspected to contain said analyte to contact component A so that analyte present in the sample interacts with component A with the generation of hydrogen peroxide;
(c) allowing the generated hydrogen peroxide to contact the reduced form of said peroxidase in the presence of ABTS so that said hydrogen peroxide is reduced and said ABTS is oxidised by said peroxidase to form ABTS_{OX}; and
(d) Allowing said ABTS_{OX} to contact said redox electrode at which it undergoes reduction to ABTS, causing an electrical current to flow in said cell; and
(e) measuring said current to provide an output signal indicative of the presence of analyte. The analyte may be determined quantitatively.

In a second aspect the invention provides a sensor for use in such a method. The sensor may be a disposable, single-use item.

In a third aspect the invention provides a method of producing such a sensor.

A preferred type of embodiment, e.g. for cholesterol measurement, employs a single-use screenprinted electrode, and preferably uses ABTS (2,2′-azino-bis-(3-ethylbenzthiazoline-6-sulfonic acid)) as an electrochemical mediator instead of as a chromogen.

This can provide three important advantages in cholesterol testing:
1) ease & economy of one-shot electrochemical testing;
2) circumvention of the well-known lack of direct electrochemical mediators for Cholesterol Oxidase (Davis, Vaughn & Cardosi, Anal. Proc. Anal. Comm., 32, 283-284, 1995);
3) reduction of most electrochemical blood interferences by enabling use of a low electrode potential.

### BRIEF DESCRIPTION OF DRAWINGS

FIG. 1 is a diagram showing the chemistry of a preferred embodiment;

FIG. 2 is a schemative view of apparatus for carrying out an embodiment;

FIG. 3 is a schemative cross section of a sensor embodying the invention;

FIG. 4 is a graph showing responses of a sensor embodying the invention; and

FIG. 5 is a glucose calibration graph for sensors embodying the invention.

### MODES FOR CARRYING OUT THE INVENTION

A preferred embodiment is a novel use of an HRP (Horseradish Peroxidase) substrate, ABTS, as a mediator in an electrochemical enzyme-electrode (henceforth, a sensor) in which HRP participates in a linked two-enzyme reaction by utilising hydrogen peroxide produced by an (analyte-oxidising) oxidase (FIG. 1). This is in contrast to the usual use of ABTS as a chromogen.

ABTS is an oxidation substrate for HRP. Its oxidation is concomitant with the reduction of hydrogen peroxide which is produced by the first enzyme (usually called an oxidase). The oxidised form of ABTS is reduced by the electrode and this reduction current provides a quantification of the analyte. In this way, the ABTS mediates between an enzymic reaction and an electrode, delivering & collecting electrons in a stoichiometric fashion.

The principal envisaged application is in the assay of cholesterol in blood although other applications are possible. ABTS enables use of a low electrode potential for detection of analytes by way of the linked two-enzyme reaction (for example 100-150 mV on screenprinted carbon; Ag/AgCl reference). Low electrode potential will preclude or reduce many of the operational interferences to be found in clinical samples such as blood (for example ascorbate, urate, acetaminophen). The linked reaction also enables electrochemical detection of other analytes which are oxidised by way of an oxidase enzyme which may not readily pass electrons directly to a mediator (as is the case with cholesterol oxidase).

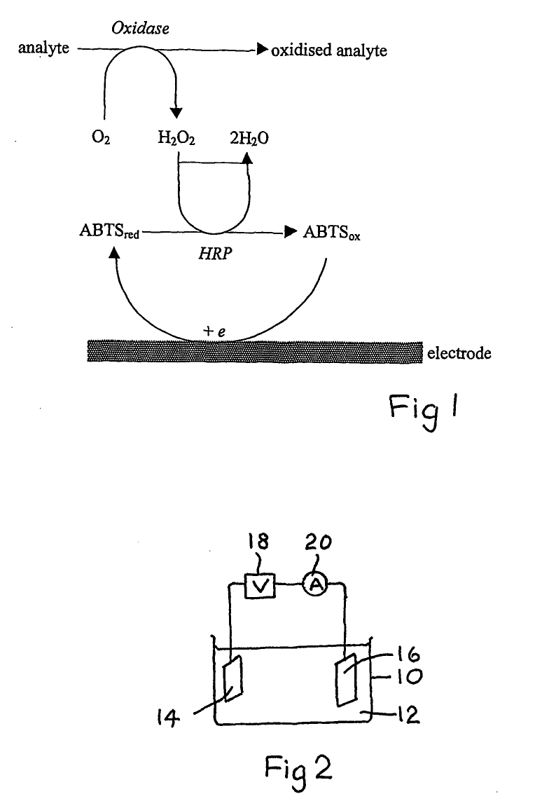

FIG. 2 is a highly schemative view of an apparatus for carrying out a method embodying the invention. A vessel 10 contains an electrolyte solution 12. A sensor electrode 14 and a counter/reference electrode 16 extend into the solution. Externally they are connected by a constant voltage source 18 and a microammeter 20. A sample for analysis is added to the solution. The necessary enzymes, mediator and any other necessary chemicals may be present in the solution or in a porous layer provided on the sensor electrode.

A more practical type of embodiment may employ a sensor electrode assembly as follows.

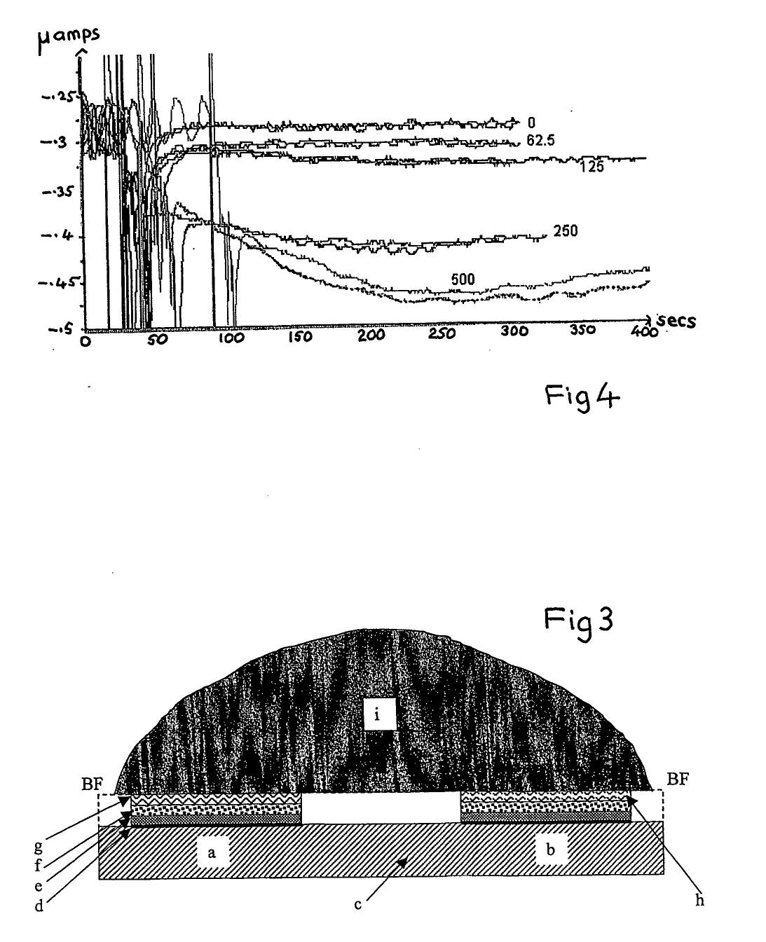

An electrode has an electrode surface. A dry layer across the electrode surface is impregnated with enzymes, ABTS, buffer & electrolyte. This layer will be hydrated and activated by addition of sample containing analyte. The layer may also contain reagents required to facilitate the solubilisation or dissolution of the sample and/or analyte. Alternatively, such reagents may be carried in a separate layer which is close to the enzyme-containing layer and through which the sample passes. Either layer may also contain materials for the selective removal, or partial removal, of interferences (such materials may also be carried in a separate layer). This removal of interferences may be by way of chemical reaction or precipitation such that the interferent species is converted to a non-interferent species or a less-interferent species or else is prevented from interfering by way of precipitation. In this respect, two examples of an interferant species are HDL- and LDL-cholesterol (high density lipoprotein & low density lipoprotein). A two-channel cholesterol sensor can be envisaged in which one channel measures total cholesterol whilst the other channel measures either HDL- or LDL-cholesterol after removal of the other (interferent) species. The HDL:LDL ratio as well as total cholesterol concentration could be calculated from the result given by the two channels when tested with the same blood sample. The layers described could be composed of pre-formed membranes or of solutions, suspensions or slurries which are deposited as layers on the electrode surface. A specific example of such a sensor would be one designed to detect cholesterol in blood as shown in FIG. 3. In this case, the enzymes are cholesterol oxidase, cholesterol esterase & HRP; detergents are incorporated to solubilise the cholesterol (examples are Triton & cholate). Cholesterol esterase is present to hydrolyse cholesterol esters. A pre-filter may be required to separate blood cells from plasma.

FIG. 3. shows a cross-section of a two-channel sensor for cholesterol in blood which utilises ABTS. The two channels, indicative by letters a and b, are mounted on a support c. Channel a detects either HDL or LDL cholesterol, channel b detects total cholesterol. Also shown are a working electrode d, a layer e containing cholesterol esterase, cholesterol oxidase, HRP and ABTS; a layer f containing components for effecting solubilisation of sample; and a layer g containing components for effecting removal of either HDL or LDL cholesterol. Channel b may contain a corresponding layer h which does not contain the agents for removal of HDL or LDL (otherwise the two channels are identical). Each channel may be isolated from the other by a support or barrier as shown by the clear area (dashed lines) if this is necessary to prevent inter-channel interference. A blood filter (at BF) may be added to filter blood cells. The blood sample is shown at i in the form of a droplet, however it could also be emplaced by capillary forces in the form of a film. Reference and (if used) counter-electrodes are placed appropriately.

It is important to note that the format shown, in which each channel is shown as three distinct layers (e, f & g), is intended only to emphasise the different functional components of the sensor. These components may also be mixed into two or even one layer.

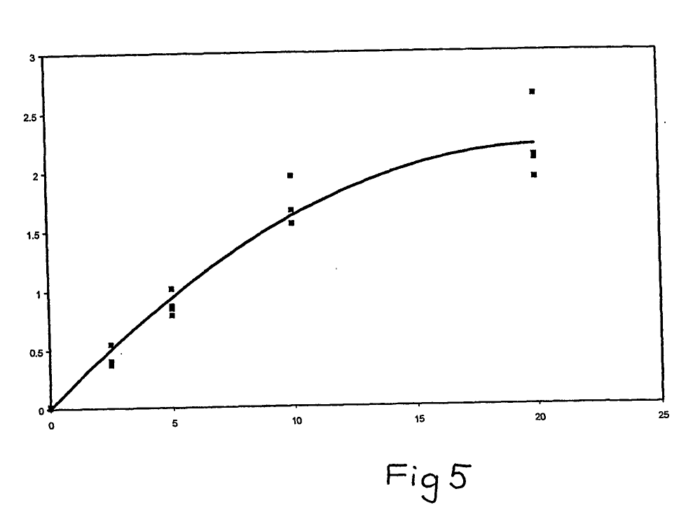

FIG. 4 shows a sample of results obtained using an impregnated cellulose membrane on top of a screenprinted carbon electrode and adding a drop of cholesterol solution. The electrode is poised at 150 mV and the cathodic current is followed. Cholesterol concentrations are shown in mg/dL. The figure shows amperometric responses of (single-channel) cholesterol sensors. Cellulose membrane discs (6 mm diameter) were impregnated with a solution of cholesterol oxidase, HRP, ABTS, buffer and electrolyte. After drying, each disc was fixed on top of a screenprinted electrode target-area (6 mm diameter) which contained working-, counter- and reference-electrode elements. The potential was poised at 150 mv (Ag/AgCl ref) and 10 μl cholesterol solution (in Triton X-100 and cholate) added to the disc; cathodic current was followed. Cholesterol concentrations are shown in mg/dL.

Another example is a sensor for blood glucose, for which the enzymes incorporated would be glucose oxidase and HRP. FIG. 5 shows a calibration curve for glucose using this sensor format.

Sensors were made and tested as described in connection with FIG. 4, except that glucose oxidase was used instead of cholesterol oxidase. Glucose solution in water (10 μl) was added to the dose. By substituting appropriate alternative oxidases (which oxidise different substrates), analagous sensors could also be formulated for detection and quantification of other analytes. These include sugars (such as galactose), carbohydrates, amino acids (such as glutamate), glycerophosphate, ethanol (and other alcohols), choline, xanthine and oxidisable carboxylic acids (i.e carboxylic acids having, in addition to carboxyl groups, functional groups rendering them more readily oxidisable than simply fatty acids, particularly oxygen-containing groups) (e.g. pyruvate, lactate and glycollate). Substitution of monoamine oxidase would allow detection of simple amines (such as methylamine, dimethylamine, trimethylamine and aminoacetone) and also more complex primary, secondary and tertiary amines, examples of which are adrenaline, serotonin, dopamine, tyramine, histamine and benzylamine. Substitution of diamine oxidase would allow detection of diamines like putrescine and cadaverine. Substitution of polyamine oxidase would allow detection of polyamines such as spermine and spermidine. Substitution of uric acid oxidase (uricase) would allow detection of uric acid (which can have pathological implications in humans).

As a corollary of this sensor format it might be envisaged that a sensor could be constructed lacking the oxidase enzyme but containing oxidase substrate. Such a sensor could then be used to detect oxidase activity in an applied sample. This could be pertinent to the monitoring of xanthine oxidase levels which can be indicative of liver pathology.

It is stressed that an alternative oxidation substrate for HRP, which is known to be electrochemically active (such as ferrocyanide), could clearly be used instead of ABTS in this application. However, this invention lies in the novel identification of ABTS as a suitable electrochemical mediator in the described sensor format.

1. A method of detecting an analyte wherein the analyte is either (a) a substrate which is oxidisable by means of an oxidase with the generation of hydrogen peroxide, the quantity of hydrogen peroxide being dependent on the quantity of analyte, or said analyte is convertible into a said oxidisable substrate, or (b) said oxidase; said method comprising:
(a) providing component A which comprises (a) said oxidase if the analyte is said oxidisable substrate, or (b) said oxidisable substrate if the analyte is said oxidase; providing component B which comprises a peroxidase capable of oxidising a mediator comprising 2,2′-azino-bis-(3-ethylbenzthiazoline-6-sulphonic acid) (“ABTS”) with concomitant reduction of hydrogen peroxide; and providing an electrochemical cell comprising a redox electrode connected to a counter-electrode;
(b) allowing a sample suspected to contain said analyte to contact component A so that analyte present in the sample interacts with component A with the generation of hydrogen peroxide;
(c) allowing the generated hydrogen peroxide to contact the reduced form of said peroxidase in the presence of ABTS so that said hydrogen peroxide is reduced and said ABTS is oxidised by said peroxidase to form ABTS_{OX}; and
(d) Allowing said ABTS_{OX} to contact said redox electrode at which it undergoes reduction to ABTS, causing an electrical current to flow in said cell; and
(e) measuring said current to provide an output signal indicative of the presence of analyte.

2. A method according to claim 1 wherein the analyte is an oxidisable substrate or is convertible into an oxidisable substrate, and component A comprises an oxidase.

3. A method according to claim 1 or claim 2 wherein the oxidisable substrate is selected from cholesterol, sugars and other carbohydrates, amino acids, glycerophosphate, alcohols, choline, xanthine, oxidisable carboxylic acids, amines and uric acid.

4. A method according to claim 3 wherein the oxidisable substrate is selected from cholesterol, glucose, galactose, glutamate, glycerophosphate, ethanol, choline, xanthine, pyruvate, lactate, glycollate, methylamine, dimethylamine, trimethylamine, aminoacetone, adrenaline, serotonin, dopamine, tyramine, histamine, benzylamine, putrescine, cadaverine, spermine, spermidine and uric acid.

5. A method according to claim 1 wherein the oxidase is selected from: cholesterol oxidase, glucose oxidase, monoamine oxidase, diamine oxidase, polyamine oxidase, uric acid oxidase and xanthine oxidase.

6. A method according to claim 1 wherein the oxidisable substrate is cholesterol and the oxidase is cholesterol oxidase.

7. A method according to claim 1 in which the analyte comprises a component which requires pre-treatment to convert it into the oxidisable substrate, and component A includes a reagent for effecting pre-treatement.

8. A method according to claim 7 in which the analyte comprises a cholesterol ester and component A includes cholesterol esterase.

9. A method according to claim 1 in which the peroxidase is horseradish peroxidase.

10. A method according to claim 1 wherein said sample is a blood sample.

11. A method according to claim 10 including a prefiltering step to separate blood cells from plasma.

12. A method according to claim 1 including a step of pre-treating a test sample to remove or render non-interfering components liable to interfere with the detection of the analyte.

13. A method according to claim 12 wherein a first portion of the test sample receives said pre-treatment and a second portion undergoes the method without said pre-treatment, and the output signals resulting from the two portions are compared.

14. A sensor device for use in carrying out the method of claim 1, comprising:
an electrode having an electrode surface;
one or more porous or otherwise permeable layers on said electrode surface containing components A and B, and a mediator comprising ABTS.

15. A sensor device according to claim 14 including an outer porous layer on said electrode to act as a filter.

16. A sensor device according to claim 14 also including in said layer or layers one or more of: buffer components; electrolyte; and components for facilitating the solubilisation and/or dissolution of a test sample or analyte therein.

17. A sensor device according to claim 14, wherein said layer or layers include one or more components for removing or rendering less active potentially interfering substrates.

18. A sensor device according to claim 16 including a first said electrode bearing said one or more porous layers, and a second said electrode bearing said one or more porous layers; wherein one of said electrodes has in its layer or layers a component for removing or rendering less active a potentially interfering substance, said component not being present at the other electrode, whereby the electrodes can provide differential outputs.

19. A sensor device according to claim 14 including a substrate and wherein said electrode is a carbon electrode screen-printed onto said substrate.

20. A method of producing a sensor device according to claim 14 comprising providing an insulating substrate, printing one or more electrodes onto said substrate; and applying over a printed electrode a layer or layers containing A, B and the mediator.

21 (Canceled).
